(19) United States
(12) Patent Application Publication
(10) Pub. No.: US 2024/0342197 A1
(43) Pub. Date: Oct. 17, 2024

(54) LIMONENE EFFECTS ON TETRAHYDROCANNABINOL (THC)-INDUCED ANXIETY

(71) Applicants: The Johns Hopkins University, Baltimore, MD (US); CReDO Science, LLC, Vashon, WA (US)

(72) Inventors: Ryan Vandrey, Baltimore, MD (US); Ethan Russo, Vashon, WA (US); Tory Spindle, Baltimore, MD (US)

(21) Appl. No.: 18/263,205

(22) PCT Filed: Jan. 28, 2022
(86) PCT No.: PCT/US22/14296

§ 371 Date: Jul. 27, 2023

Related U.S. Application Data
(60) Provisional application No. 63/143,347, filed on Jan. 29, 2021.

(51) Int. Cl. A61K 31/00; A61K 31/015; A61P 25/22

(52) U.S. Cl. CPC A61K 31/658; A61K 31/015; A61P 25/22

(57) ABSTRACT

Non-naturally occurring compositions comprising tetrahydrocannabinol (THC) and limonene and their use for treating THC-induced anxiety and/or paranoia are disclosed.

### STATEMENT OF GOVERNMENTAL INTEREST

This invention was made with government support under grant DA043475 awarded by the National Institutes of Health. The government has certain rights in the invention.

### BACKGROUND

Tetrahydrocannabinol (THC) has been the primary focus of cannabis research since its isolation and synthesis in 1964 (Gaoni and Mechoulam 1964). Currently, THC is approved by the U.S. Food and Drug Administration (FDA) for multiple therapeutic conditions and is used “off-label” for a multitude of conditions through state-administered medical cannabis programs. Whether non-THC constituents of the cannabis plant contribute to the overall pharmacodynamic effects of cannabis, and which non-THC constituents may do so, also has been under debate. Recent publications have hypothesized that specific cannabis terpenoids, aromatic essential oil (EO) components, may selectively mitigate or exacerbate some acute effects of THC (McPartland and Russo 2001, Russo 2011, McPartland and Russo 2014). This purported “entourage effect” has been the driving force behind state legalization of cannabis for medicinal purposes despite THC (as dronabinol) being available as an FDA-approved medication. There have been no empirical research studies conducted, however, to evaluate the interactions of THC and terpenoids found in the cannabis plant.

### SUMMARY

In some aspects, the presently disclosed subject matter comprises a non-naturally occurring composition comprising a combination of tetrahydrocannabinol (THC) and limonene. In certain aspects, the THC comprises a synthetic THC. In particular aspects, the synthetic THC comprises dronabinol.

In some aspects, the THC and limonene are present in the composition in a ratio of about 2:1 (THC:limonene). In certain aspects, the composition comprises between about 1 mg to about 50 mg THC. In certain aspects, the composition comprises between about 0.5 mg to about 25 mg limonene. In particular aspects, the composition of THC and limonene is selected from the group consisting of 15 mg THC:1 mg limonene; 30 mg THC:1 mg limonene; 15 mg THC:5 mg limonene; 30 mg THC:5 mg limonene; and 30 mg THC:15 mg limonene.

In some aspects, the composition further comprises a pharmaceutically acceptable carrier. In certain aspects, the pharmaceutically acceptable carrier is selected from the group consisting of propylene glycol, glycerin, and sesame seed oil.

In some aspects, the composition is formulated in a gelcap. In certain aspects, the composition comprises from about 1 mg to about 30 mg of THC and from about 1 mg to about 1,000 mg of limonene. In other aspects, the composition comprises a formulation for oromucosal delivery. In such aspects, the formulation for oromucosal delivery comprises a medium-chain triglyceride (MCT) oil, a breath strip, or an oromucosal spray.

In other aspects, the presently disclosed subject matter provides a method for reducing or mitigating paranoia and/or one or more anxiogenic effects of tetrahydrocannabinol (THC), the method comprising administering to a subject in need of treatment thereof the presently disclosed non-naturally occurring composition of THC and limonene. In certain aspects, the one or more anxiogenic effects comprises THC-induced anxiety. In other aspects, the method reduces or mitigates THC-induced paranoia.

In some aspects, the administering the combination of THC and limonene lowers a level of THC-induced anxiety and/or paranoia in the subject relative to a level of THC-induced anxiety and/or paranoia experienced by the subject when administered THC alone.

In some aspects, the non-naturally occurring composition comprising a combination of tetrahydrocannabinol (THC) and limonene is administered orally, by vaporization, or via oromucosal delivery.

In other aspects, the presently disclosed subject matter provides a method for treating one or more conditions or symptoms in a subject in need of treatment thereof, the method comprising administering a therapeutically effective amount of the presently disclosed non-naturally occurring composition of THC and limonene to the subject to treat the one or more conditions.

In some aspects, the one or more conditions or symptoms is selected from the group consisting of arthritis, osteoarthritis, arthralgia, joint pain, joint stiffness, diabetes, lack of appetite, anorexia, vomiting, nausea, an inflammatory bowel disease including Crohn's disease and ulcerative colitis, dementia, memory loss, osteoporosis, fatigue, weakness, decreased mental energy, decreased physical energy, dizziness, deficits in balance, itchy skin, pruritus and/or chronic pruritus, wrinkles, stretch marks, skin burn, headache, migraine, weight gain, digestive problem, intestinal disorder, gastrointestinal pain, abdominal pain, pelvic pain, constipation, diarrhea, hot flashes, sweating, difficulty in concentration, weakened immune system, cardiovascular disease, palpitation and tachycardia, psoriasis, dermatophytes, Candida, leishmaniasis, Methicillin-resistant Staphylococcus aureus (MRSA), malaria, allergy, fibromyalgia, nociceptive pain, neuropathic pain, pain, arm or leg pain, acne, insomnia and/or sleep disorders, muscle pain, myalgia, spasticity, muscle tension, cramps, spasms, anxiety, irritability, nervousness, restless, stress, depression, mood problem, affect disorders, anger, autism and/or autism spectrum disorder, neurodegenerative diseases, Alzheimer disease, Parkinson disease, Huntington's disease, Dystonia, Amyotrophic Lateral Sclerosis (ALS), multiple sclerosis, Tourette syndrome, Myasthenia Gravis, epilepsy, symptomatic treatment of cancer, primary treatment of cancer, inflammatory airway diseases including asthma and/or chronic obstructive pulmonary disease (COPD), addiction, stroke, traumatic brain injury, motor spasm and/or tic, vocal tic, obsessive compulsive disorder (OCD), attention deficit hyperactivity disorder (ADHD), attention deficit disorder (ADD), cerebral palsy (CP), inflammation, oxidative stress, paresthesia, glaucoma, heartburn, pyrosis, gastric ulcers and H. pylori infection, and combinations thereof.

In certain aspects, the one or more conditions or symptoms is selected from the group consisting of anorexia associated with weight loss in subjects afflicted with AIDS, nausea and vomiting associated with cancer chemotherapy in subjects who have failed to respond to conventional antiemetic treatments, and sleep apnea.

In some aspects, the non-naturally occurring composition comprising a combination of tetrahydrocannabinol (THC) and limonene is administered orally, by vaporization, or via oromucosal delivery.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1 shows plots of the Visual Analogue Scale (VAS) for Anxiety versus time for a 10-mg dose of THC (left panel) and a 25-mg dose of THC (right panel) via smoke, vaporized, or oral administration; and

FIG. 2 shows triplicate testing of dose-response of limonene as measured by GC/MS of either direct injection into the Volcano Medic balloons or vaporized at 210° C. using the Volcano Medic;

FIG. 3 is a plot of the Visual Analogue Scale (VAS) for Anxiety for dosages of a placebo, 1 mg limonene, 5 mg limonene, 15 mg THC, 15 mg THC+1 mg limonene, 15 mg THC+5 mg limonene, 30 mg THC, 30 mg THC+1 mg limonene, 30 mg THC+5 mg limonene, and 30 mg THC+15 mg limonene (n=16);

FIG. 4 is a plot of the Visual Analogue Scale (VAS) for Paranoia for dosages of a placebo, 1 mg limonene, 5 mg limonene, 15 mg THC, 15 mg THC+1 mg limonene, 15 mg THC+5 mg limonene, 30 mg THC, 30 mg THC+1 mg limonene, 30 mg THC+5 mg limonene, and 30 mg THC+15 mg limonene (n=16); and

FIG. 5 is a plot of the Visual Analogue Scale (VAS) for Drug Effect for dosages of a placebo, 1 mg limonene, 5 mg limonene, 15 mg THC, 15 mg THC+1 mg limonene, 15 mg THC+5 mg limonene, 30 mg THC, 30 mg THC+1 mg limonene, 30 mg THC+5 mg limonene, and 30 mg THC+5 mg limonene.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed. many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Limonene Effects on Tetrahydrocannabinol (THC)-Induced Anxiety

The presently disclosed subject matter, in part, evaluates the interaction between tetrahydrocannabinol (THC), the primary psychoactive constituent of the cannabis plant, and limonene, a terpenoid that is abundant in many plant species. The presently disclosed subject matter demonstrates that limonene can mitigate the anxiogenic effects and/or paranoia associated with acute high doses of THC. In some embodiments, the presently disclosed subject matter evaluates the acute dose effects of THC and d-limonene (limonene) alone and in combination. The presently disclosed subject matter has broad appeal for the development of THC products in which the addition of limonene could significantly reduce adverse effects and make the drug more tolerable.

In some embodiments, the presently disclosed subject matter comprises a non-naturally occurring composition comprising a combination of tetrahydrocannabinol (THC) and limonene. In certain embodiments, the THC comprises a synthetic THC. In particular embodiments, the synthetic THC comprises dronabinol.

In some embodiments, the THC and limonene are present in the composition in a ratio of about 2:1 (THC:limonene), including about 3:1, 2.9:1, 2.8:1, 2.7:1, 2.6:1, 2.5:1, 2.4:1, 2.3:1, 2.2:1, 2.1:1, 2.0:1, 1.9:1, 1.8:1, 1.7:1, 1.6:1, and 1.5:1. In certain embodiments, the composition comprises between about 1 mg to about 50 mg THC, including about 1, 2, 3, 4, 5, 6, 7, 8, 9, 10, 11, 12, 13, 14, 15, 16, 17, 18, 19, 20, 21, 22, 23,24,25,26,27,28,29,30,31,32,33,34,35,36,37,38,39,40,41,42,43,44,45,46, 47, 48, 49, and 50 mg THC. In certain embodiments, the composition comprises between about 0.5 mg to about 25 mg limonene, including about 0.5, 1, 2, 3, 4, 5, 6, 7, 8, 9, 10, 11, 12, 13, 14, 15, 16, 17, 18, 19, 20, 21, 22, 23, 24, and 25 mg limonene. In particular embodiments, the composition of THC and limonene is selected from the group consisting of 15 mg THC:1 mg limonene; 30 mg THC:1 mg limonene; 15 mg THC:5 mg limonene; 30 mg THC:5 mg limonene; and 30 mg THC:15 mg limonene.

In some embodiments, the composition further comprises a pharmaceutically acceptable carrier. In certain embodiments, the pharmaceutically acceptable carrier is selected from the group consisting of propylene glycol, glycerin, and sesame seed oil.

In other embodiments, the presently disclosed subject matter provides a method for reducing or mitigating paranoia and/or one or more anxiogenic effects of tetrahydrocannabinol (THC), the method comprising administering to a subject in need of treatment thereof the presently disclosed non-naturally occurring composition of THC and limonene. In certain embodiments, the one or more anxiogenic effects comprises THC-induced anxiety. In other embodiments, the method reduces or mitigates THC-induced paranoia.

In some embodiments, the administering the combination of THC and limonene lowers a level of THC-induced anxiety and/or paranoia in the subject relative to a level of THC-induced anxiety and/or paranoia experienced by the subject when administered THC alone.

In some embodiments, the non-naturally occurring composition comprising a combination of tetrahydrocannabinol (THC) and limonene is administered orally, by vaporization, or via oromucosal delivery.

In other embodiments, the presently disclosed subject matter provides a method for treating one or more conditions or symptoms in a subject in need of treatment thereof, the method comprising administering a therapeutically effective amount of the presently disclosed non-naturally occurring composition of THC and limonene to the subject to treat the one or more conditions.

In some embodiments, the one or more conditions or symptoms is selected from the group consisting of arthritis, osteoarthritis, arthralgia, joint pain, joint stiffness, diabetes, lack of appetite, anorexia, vomiting, nausea, an inflammatory bowel disease including Crohn's disease and ulcerative colitis, dementia, memory loss, osteoporosis, fatigue, weakness, decreased mental energy, decreased physical energy, dizziness, deficits in balance, itchy skin, pruritus and/or chronic pruritus, wrinkles, stretch marks, skin burn, headache, migraine, weight gain, digestive problem, intestinal disorder, gastrointestinal pain, abdominal pain, pelvic pain, constipation, diarrhea, hot flashes, sweating, difficulty in concentration, weakened immune system, cardiovascular disease, palpitation and tachycardia, psoriasis, dermatophytes, Candida, leishmaniasis, Methicillin-resistant Staphylococcus aureus (MRSA), malaria, allergy, fibromyalgia, nociceptive pain, neuropathic pain, pain, arm or leg pain, acne, insomnia and/or sleep disorders, muscle pain, myalgia, spasticity, muscle tension, cramps, spasms, anxiety, irritability, nervousness, restless, stress, depression, mood problem, affect disorders, anger, autism and/or autism spectrum disorder, neurodegenerative diseases, Alzheimer disease, Parkinson disease, Huntington's disease, Dystonia, Amyotrophic Lateral Sclerosis (ALS), multiple sclerosis, Tourette syndrome, Myasthenia Gravis, epilepsy, symptomatic treatment of cancer, primary treatment of cancer, inflammatory airway diseases including asthma and/or chronic obstructive pulmonary disease (COPD), addiction, stroke, traumatic brain injury, motor spasm and/or tic, vocal tic, obsessive compulsive disorder (OCD), attention deficit hyperactivity disorder (ADHD), attention deficit disorder (ADD), cerebral palsy (CP), inflammation, oxidative stress, paresthesia, glaucoma, heartburn, pyrosis, gastric ulcers and H. pylori infection, and combinations thereof. For example, cannabis extract has been shown to be effective for treating duodenal ulcers. See, for example, Douthwaite, A. H., Choice of Drugs in the Treatment of Duodenal Ulcer, British Medical Journal, 4514: 43-47 (1947).

In certain embodiments, the one or more conditions or symptoms is selected from the group consisting of anorexia associated with weight loss in subjects afflicted with AIDS, nausea and vomiting associated with cancer chemotherapy in subjects who have failed to respond to conventional antiemetic treatments, and sleep apnea.

In some embodiments, the non-naturally occurring composition comprising a combination of tetrahydrocannabinol (THC) and limonene is administered orally by vaporization, or via oromucosal delivery.

In certain embodiments, the non-naturally occurring composition comprising a combination of tetrahydrocannabinol (THC) and limonene is in the form of a large gel cap for oral administration. In such embodiments, the composition can comprise from about 1 mg to about 30 mg of THC, including about 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 mg THC. Further, in such embodiments, the composition can comprise from about 1 to about 1,000 mg of limonene, including about 1, 2, 3, 4, 5, 6, 7, 8, 9, 10, 11, 12, 13, 14, 15, 20, 25, 50, 100, 200, 300, 400, 500, 600, 700, 800, 900, and 1000 mg of limonene, including any integers and fractions thereof throughout the recited range. As used herein the term “gelcap” refers to a capsule-shaped shell comprising or coated with gelatin, which encapsulates the therapeutic agent, i.e., the presently disclosed composition of THC and limonene. For example, a soft-shelled capsules can be used for oils and for active ingredients that are dissolved or suspended in oil. Such capsules can include gelling agents, such as animal protein, e.g., gelatin, or plant polysaccharides or derivatives thereof, such as carrageenans and modified forms of starch and cellulose. Other ingredients can be added to the gelling agent solution including plasticizers, such as glycerin or sorbitol, to decrease the capsule's hardness, coloring agents, preservatives, disintegrants, lubricants, other surface treatments, and the like.

In yet other embodiments, the non-naturally occurring composition comprising a combination of tetrahydrocannabinol (THC) and limonene is administered via oromucosal delivery, which may allow better absorption and efficacy of the d-limonene component. Representative embodiments for oromucosal delivery include, but are not limited to, medium-chain triglyceride (MCT) oil, breath strips, oromucosal spray, and the like. Such oromucosal sprays can include the presently disclose composition dispersed in alcohol or other suitable solvent and can be administered under the tongue or on the buccal mucosa.

As used herein, the term “treating” can include reducing, reversing, alleviating, inhibiting the progression of, mitigating, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly THC and limonene. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, THC and limonene can be administered alone or in combination with adjuvants that enhance stability of the composition thereof, facilitate their administration, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of THC and limonene can be varied so long as the beneficial effects of the combination of these agents are achieved. Accordingly, the phrase “in combination with” refers to the administration of THC and limonene either simultaneously, sequentially, or a combination thereof. Therefore, a subject administered a combination of THC and limonene can receive the THC and limonene at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where THC and limonene are administered simultaneously, they can be administered to the subject as separate pharmaceutical compositions, each comprising either THC or limonene, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of THC and limonene is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( S I )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Limonene Effects on Tetrahydrocannabinol (THC)-Induced Anxiety

Delta-9-tetrahydrocannabinol (THC) is the primary psychoactive chemical constituent of the cannabis plant. The effects of THC have been well characterized in controlled research. Positive and/or therapeutic effects include feelings of euphoria, relaxed mood, enhanced enjoyment of music/art, as well as analgesic, anti-inflammatory, hypnotic, muscle relaxant, bronchodilatory, antiemetic, and appetite stimulant effects.

Negative or unwanted side effects include dysphoria (e.g., panic, paranoia, and acute psychosis), nausea/emesis dry mouth, irritated eyes, hallucinations, and cognitive impairment (e.g., working memory, divided attention, time estimation, and complex cognition). These effects are produced through a combination of partial agonism at the CB₁ and CB₂ receptors, as well as non-receptor mechanisms (Russo 2011).

For many, THC is synonymous with cannabis, and, over the past 30 years, illicit drug producers have selectively bred cannabis plants to contain ever-greater concentrations of THC, which now accounts for 15-25% of the dried flowers of the plant sold to consumers in the U.S. Also, with the advent of a legal medicinal and non-medicinal cannabis market in over half the U.S. states, a larger emerging product market of “concentrates” has developed. In production of these products, THC is extracted from the plant material resulting in a resin that contains 75-90% THC. Recently, a trend has emerged among manufacturers to “spike” these cannabis resins with select terpenoids or terpenoid combinations as a means of producing tailored pharmacodynamic effects.

One issue in understanding the pharmacology of cannabis is whether its behavioral and psychoactive effects are wholly accounted for by THC, or whether other cannabis components, including “minor” cannabinoids (e.g., cannabidiol, cannabinol, and cannabigerol) and terpenoids substantively influence its effects. This issue has been compounded by the fact that cannabis supplied for experimentation in the U.S. is notably deficient in minor cannabinoid and terpenoid content as compared to that available via the black market, where plants are selectively bred to have specific cannabinoid and terpenoid profiles based on the belief that certain ratios confer different effects on the user (Bloor et al. 2008). These beliefs, however, are based largely on anecdote, with little controlled research to inform the interaction of THC and minor cannabinoids and no human research on the interaction between THC and terpenoids has been published to date.

Terpenoids are produced in glandular trichomes of cannabis along with phytocannabinoids. Terpenoids are pharmacologically versatile, including interacting with cell membranes, neuronal and muscle ion channels, neurotransmitter receptors, G-protein coupled (odorant) receptors, second messenger systems and enzymes (Bowles 2003, Buchbauer 2010). One such terpenoid is d-limonene (also referred to herein as “limonene”). Limonene is a flavor and fragrance component common to many plants and is a fragrance ingredient in many household products. Because limonene is present in most citrus fruits, it is part of the typical human diet. Oral ingestion of d-limonene is Generally Recognized as Safe (GRAS) by the FDA and other regulatory agencies.

D-limonene is common to the lemon and other citrus essential oils (EOs) and is the second most widely distributed terpenoid in nature (Noma and Asakawa 2010). D-limonene also is one of the most abundant terpenoids found in the cannabis plant. Studies have been conducted evaluating limonene's effects when inhaled in ambient air. For example, prior research has demonstrated that limonene inhaled from ambient air can produce anxiolytic effects and reduce depression (Buchbauer et al. 1993, Carvalho-Freitas and Costa 2002a, Pultrini Ade, Galindo, and Costa 2006, Falk-Filipsson et al. 1993).

Studies of citrus oils in mice suggest d-limonene produces anxiolytic effects (Carvalho-Freitas and Costa 2002b). The mechanism of these effects is hypothesized to be increased serotonin in prefrontal cortex (PFC), and dopamine (DA) in hippocampus mediated via 5-HT_{1A} receptors (Komiya, Takeuchi, and Harada 2006). Human research on the anxiolytic effects of inhaled d-limonene has been limited to a single study (Komori et al. 1995), in which hospitalized depressed patients were exposed to citrus fragrance in ambient air. In that study, Hamilton Depression Scores (HADS) were reduced by the citrus fragrance, 9/12 patients discontinued antidepressant medication, and serum evidence of immune stimulation (CD4/8 ratio normalization) was observed. Inhalation of d-limonene has high bioavailability, with an estimated 70% human pulmonary uptake, and was well tolerated in a controlled exposure study (Falk-Filipsson et al. 1993). D-limonene produces a hepatic metabolite, perillic acid, which demonstrates stress reduction effects in the rat brain (Fukumoto et al. 2008). D-limonene is non-toxic at low doses (estimated human lethal dose 0.5-5 g/kg⁻¹) and non-sensitizing (Von Burg 1995), and an estimate of exposure to limonene for the general population via indoor air is 10 g/kg/day (Kim et al. 2013).

Currently, there is rapidly growing interest in the development of cannabis and cannabinoid-based medicines for the treatment of myriad health conditions. Dronabinol is a synthetic form of (−)-trans-Δ⁹-tetrahydrocannabinol and does not include any other tetrahydrocannabinol (THC) isomers or any cannabidiol. It is FDA approved for HIV/AIDS-induced anorexia and chemotherapy-induced nausea and vomiting. Although THC (dronabinol) has been approved by the FDA for 30 years, it is rarely used in medical practice.

Part of the reason for dronabinol's limited medicinal use is that the therapeutic index of pure THC, when given intravenously (D'Souza et al. 2004) or orally (Favrat et al. 2005), is narrow, especially among individuals previously naïve to its effects. Acute overdose incidents involving THC or THC-predominant cannabis commonly include anxiety, panic reactions or toxic psychoses, for which no pharmacological intervention is generally necessary, but which can result in significant and sustained discomfort to the individual. In recent years, advocates for cannabis legalization have argued that the use of cannabis (versus pure THC) reduces the rate and severity of adverse effects in the treatment of medical conditions. This argument has been the basis for establishing medicinal cannabis laws in 29 of the 50 U.S. states and the District of Columbia.

To date, scientific evaluation of the “entourage” effects of cannabis versus THC alone has been largely limited to the study of cannabidiol (CBD) as a compound purported to attenuate some of the untoward psychoactive effects of THC and generally reduce its adverse event profile. Clinical research by a UK pharmaceutical company suggests that producing cannabinoid medicine with a 1:1 ratio of THC:CBD produces better clinical results with fewer side effects relative to higher THC:CBD ratios (Russo and Guy 2006). Laboratory studies, however, have failed to detect a modulatory effect of CBD on the subjective effects of THC (Ilan et al. 2005, Haney et al. 2016). Though there is a lack of controlled research, numerous reports in published literature dating back to the 10^{th} century indicate that the consumption of lemons, lemonade, citrus fruits, pine nuts, calamus root, or other natural sources of d-limonene or related substances can have the effect of an “antidote” to the harms or adverse effects associated with the consumption of cannabis or hashish. Russo, 2011.

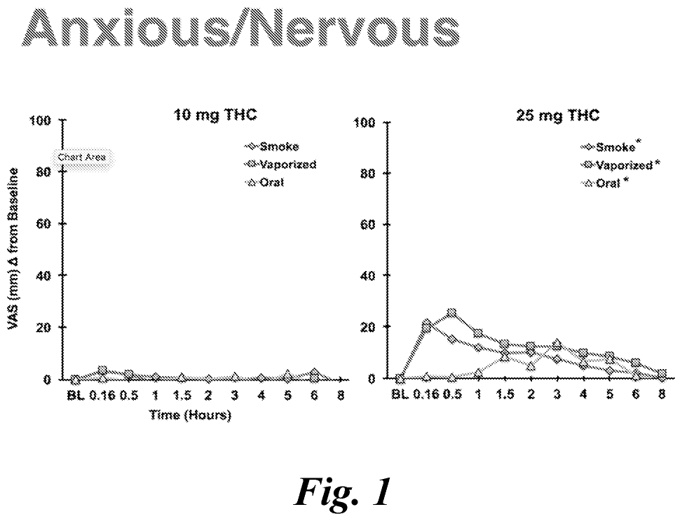

The presently disclosed subject matter is directed to evaluating, in a controlled laboratory experiment, whether and to what degree, d-limonene modulates the acute effects of inhaled THC. Anxiety, for example, is a common adverse effect of THC and cannabis products broadly. The procedures used herein were established during recent studies evaluating the dose effects of cannabis administered via oral ingestion, smoking, or vaporization. Results of those studies demonstrated that acute cannabis effects can be reliably produced and validly measured. These previous studies were used to inform the THC doses selected for this study. For example, vaporized cannabis containing THC doses of 10 mg and 25 mg could be safely administered to healthy adult volunteers. These doses produced dose-orderly drug effects. The 10-mg THC dose was not associated with an increase in anxiety and produced mild to moderate drug effects, whereas the 25-mg THC dose produced mild to moderate anxiety in a subset of subjects (see FIG. 1). It is thought that increasing the doses of THC to 15 mg and 30 mg will increase the likelihood of anxiety following dosing, as well as the severity of anxiety after dosing in the subjects, but the level of anxiety will remain tolerable.

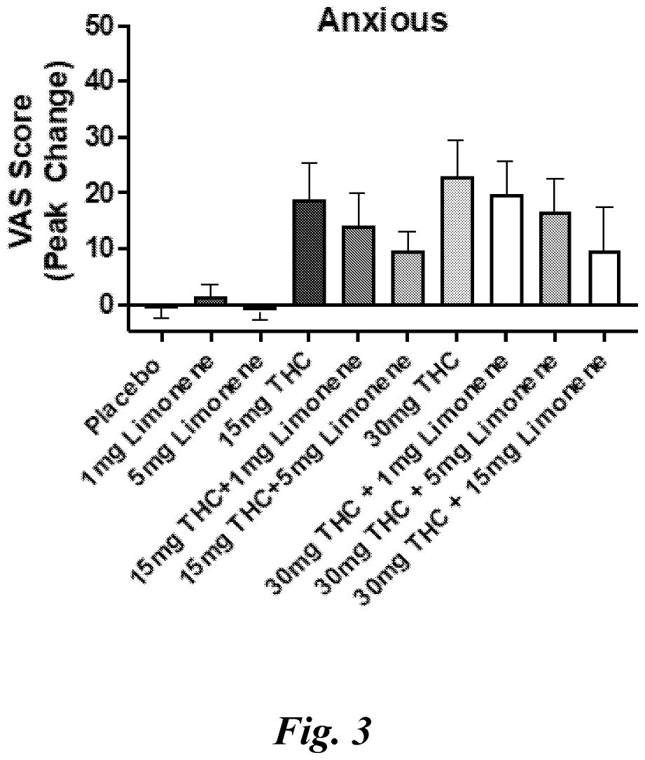

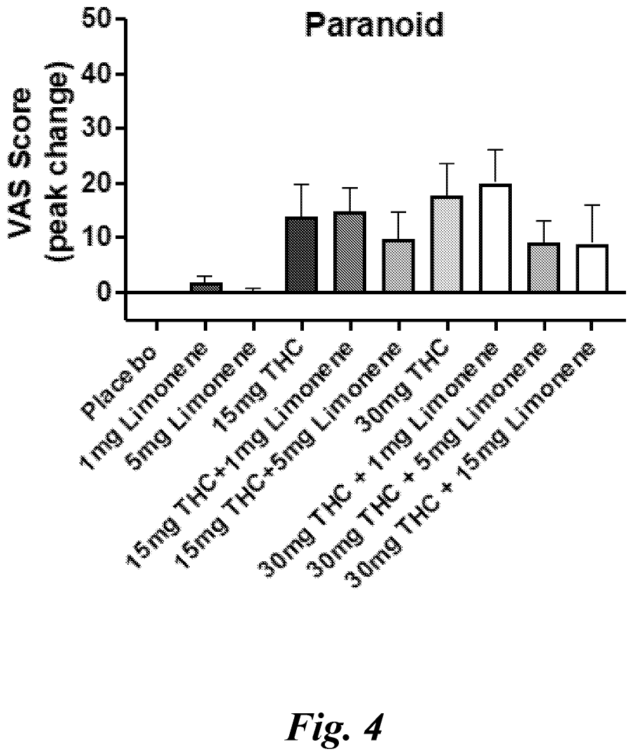

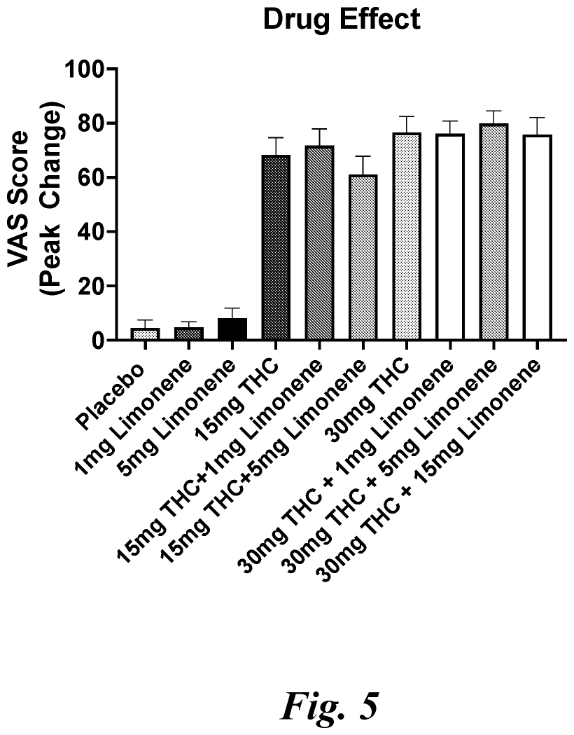

For example, referring now to FIG. 3 and FIG. 4, the mean peak subjective ratings (+SEM, change-from-baseline) for “anxious” and “paranoid” (N=16) are shown. THC alone reliably increased ratings for feeling “anxious” and “paranoid.” Ratings for these two items were reduced in a dose-dependent manner when limonene also was administered. FIG. 5 is a plot of the Visual Analogue Scale (VAS) for drug effect for dosages of a placebo, 1 mg limonene, 5 mg limonene, 15 mg THC, 15 mg THC+1 mg limonene, 15 mg THC+5 mg limonene, 30 mg THC, 30 mg THC+1 mg limonene, 30 mg THC+5 mg limonene, and 30 mg THC+5 mg limonene.

In some embodiments, the dose of d-limonene administered ranges from about 1 mg to about 30 mg, including 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 mg. In some embodiments, the dose of d-limonene is between about 1 mg to about 5 mg; in other embodiments, between about 5 mg to about 10 mg; in other embodiments, between about 10 mg to about 15 mg; in other embodiments, between about 15 mg to about 20 mg; in other embodiments, between about 20 mg to about 25 mg; and in yet other embodiments, between about 25 mg to about 30 mg. The lower end of this range, e.g., between about 1 mg to about 5 mg, is approximately an amount that would be inhaled by an individual who smokes about one gram of cannabis. In recent research studies, one gram about is the amount of plant material commonly used to make a single cannabis cigarette (“joint” or “blunt”). In addition, chemical analysis of 107 samples of cannabis consisting of 29 different “strains” of cannabis produced by a Canadian medicinal cannabis manufacturer indicate that one mg would be the median and 5 mg would be the maximum d-limonene dose in a one-gram cannabis cigarette produced with their products. Thus, these lower doses are ecologically relevant and have demonstrated safety with respect to acute dosing via direct inhalation.

The higher doses of d-limonene, e.g., the 15-mg dose, is higher than what would typically be consumed in natural botanical cannabis, but should not present any risk of harm. A current practice in the cannabis industry is to supplement formulated cannabis products with extracted terpenes. Further evidence for the safety of d-limonene comes from a prior study (Falk-Filipsson et al., 1993) in which healthy adults were continuously exposed to high levels of d-limonene in an enclosed chamber for 2 hours; in this study, subjects absorbed up to about 26 mg of d-limonene without experiencing adverse events or irritative symptoms. In preliminary studies conducted by the inventors, no adverse effects were reported following administration of either 1-mg or 5-mg d-limonene.

The presently disclosed subject matter evaluates whether limonene, compared with placebo, attenuates increases in anxiety that often occur following acute THC administration. In some embodiments, dosage regimens include: placebo (5-mL distilled water); 15 mg THC; 30 mg THC; 1 mg d-limonene; 5 mg d-limonene; 15 mg THC+1 mg d-limonene; 30 mg THC+1 mg d-limonene; 15 mg THC+5 mg d-limonene; 30 mg THC+5 mg d-limonene; and 30 mg THC+15 mg d-limonene. In some embodiments, the administration is via vaporization. In some embodiments, the number of drug administration sessions is 9.

It is expected that the presently disclosed subject matter will represent a significant advancement in understanding the behavioral pharmacology of cannabis, in which the interactions of multiple components of the cannabis plant are systematically evaluated. Characterization of the interaction between THC and d-limonene will provide a scientific basis for whether or not there is benefit for including terpenoids in the development of cannabinoid-based pharmaceutical products as one means of reducing the incidence and/or severity of side effects, including anxiety.

Accordingly, in some embodiments, the presently disclosed subject matter investigates the behavioral pharmacology of THC, d-limonene, and their combination versus placebo. In particular, the presently disclosed subject matter investigates the effect of placebo, THC only, and THC in combination with d-limonene at various dosage ranges, in subjects with a history of having experienced mild to moderate anxiety following acute cannabis exposure. To determine these effects, a battery of pharmacodynamic outcome measures at baseline and for a period of time, e.g., 6 hours, after each dose can be obtained. Baseline assessments include 5 mL serum blood sample, vital signs (heart rate (HR), blood pressure (BP)), subjective drug effect questionnaire, and a brief cognitive performance battery. A battery of subjective, physiological, and cognitive performance assessments will be completed and biological specimens obtained to determine the effect of limonene in subjects who exhibit increased anxiety after THC exposure.

The presently disclosed compositions can be administered orally, by smoking, or via vaporization, using, for example, the Mighty Medic (Storz-Bickel, Tuttlingen, Germany), a commercial vaporizer designed specifically for the delivery of cannabis and THC. In some embodiments, vaporization is preferred as the route of administration because both THC and limonene have good pulmonary bioavailability, it allows for more precise dose delivery versus smoked or oral routes, this method is most likely to protect the blind of drug conditions between sessions as there are fewer sensory cues associated with inhalation of vapor versus smoked cannabis, inhalation is the most common method of consuming cannabis, and because vaporization has the same pharmacokinetics, but less pulmonary risk, compared with smoking. The Mighty Medic employs hot air at a temperature of 210° C. to vaporize THC and terpenoids without combustion, thereby limiting exposure to potentially carcinogenic polyaromatic hydrocarbons, ammonia, and other toxins. Each drug dose is placed in a small dosing capsule (or “pod”). Subjects inhale the contents of the capsule by inhaling though a mouthpiece attached to the vaporizer, which activates the heating element and delivers the study drug. The Mighty Medic is an approved medical device in the European Union, Canada and Israel and functions similarly to the Volcano Medic, which has been used previously in by the inventors to administer raw cannabis via vaporization. The Mighty Medic and Volcano Medic are made by the same company: Storz-Bickel. Analytical testing conducted by Research Triangle Institute and others has demonstrated that the Volcano Medic reliably and dose-dependently delivers THC and d-limonene. Importantly, analytical testing conducted by Stortz and Bickel has revealed that the Volcano Medic and Mighty Medic are equally effective at delivering THC in vapor. Currently, there are no regulatory limits in the U.S. on the allowable concentration of d-limonene in ambient air, and the total exposure during an acute dose session in this study is likely not more than what one would be exposed to peeling an orange or walking down an isle of cleaning supplies at a grocery store.

A pharmacist or other qualified technician will apply test substances with a micro-pipette into a dosing capsule, accessories that come with the Mighty Medic. For each experimental session, a dosing capsule with the assigned dose will be provided for subject self-administration using the Mighty Medic in accordance with the manufacturers operating instructions. When the Mighty Medic is activated, the dosing capsule is heated, vaporizing the substances placed inside it.

At each session, study personnel will first purge residual ethanol in the Mighty Medic in an unheated state. Detailed instructions for use of the device are available here: www.storz-bickel.com/media/wysiwyg/CRAFTY-MIGHTY/PDF/mighty-vaporizer-instructions-manual.pdf. Subjects will then inhale the contents of one Mighty Medic dosing capsule. Specifically, they will be given 15 minutes to inhale the contents of the capsule ad libitum (i.e., at their own pace). Subjects will take a minimum of 15 puffs. If a visible vapor is still observed after 15 puffs, the will continue to take puffs until they no longer exhale visible vapor (this observation signifies that the dosing capsule is depleted; see detailed instructional materials from Stortz and Bickel). This procedure will produce THC exposure of about 0-mg, 15-mg, or 30-mg THC, and about 1-mg, 5-mg, or 15-mg d-limonene. Subjects will be able to halt self-administration of the study drug if they experience adverse effects prior to inhaling the entire dose. Similarly, study staff may halt the drug administration procedure if untoward drug effects are observed. This is not expected at the doses under investigation, but may occur in rare circumstances. Study subjects and research staff will be blinded to dose assignment. The conclusion of drug administration will be considered the “0 hour” by which remaining protocol assessments will be scheduled. To prevent issues of cross-contamination, new inhalation bags and thoroughly cleaned stainless steel wool pads will be utilized for every test session.

Following the “0-hour” time point (last exhalation of study drug), subjects will complete a battery of assessments including:
1) Serum specimen collection at 0 min, 15 min, 60 min, 120 min, and 180 min;
2) Subjective drug effect ratings on computerized questionnaires and vital signs assessments at 0 min, 30 min, 60 min, 90 min, 120 min, 180 min, 240 min, 300 min, and 360 min;
3) Subjective ratings of mood at 0, 30 min, 60 min, 90 min, 120 min, 180 min, 240 min, 300 min, and 360 min; and
4) Cognitive performance tests at 0, 30 min, 60 min, 90 min, 120 min, 180 min, 240 min, 300 min, and 360 min.

Further, a battery of measures will be used to assess participant characteristics and drug effects during the study. For example, vital signs (heart rate (HR), systolic blood pressure (SBP), diastolic blood pressure (DBP)) will be measured in the seated position using an automated monitor.

Six milliliters of blood will be collected by venipuncture or IV catheter insertion at baseline and again at the 0-, 15-, 60-, 120-, and 180-minute post-inhalation time points into vacutainer tubes. Blood will be spun to separate plasma, which will be labeled and stored frozen at −80° C. until shipped frozen on dry ice to a designated laboratory for analysis. The maximum amount of blood to be collected is 36 mL per session, and 324 mL over the course of the entire study, which is less than the amount typically collected during a single routine blood donation (473 mL). Quantitative levels of THC and its metabolites (e.g., 11-OH-THC and THCCOOH) will be obtained. Additional analytical testing (e.g., markers of limonene or stress) may be conducted as deemed appropriate to the current study.

A 22-item Drug Effect Questionnaire will be used to obtain subjective ratings of intoxication. Individual items include ratings of drug effects (i.e., drug effect, pleasant drug effect, and unpleasant drug effect) and behavioral/mood states often associated with cannabis intoxication (i.e., relaxed, paranoid, and hungry/have munchies). Subjects will rate each item using a 100-mm visual analog scale (VAS) anchored with “not at all” on one end and “extremely” on the other.

The 20-item State subscale of the State-Trait Anxiety Inventory (STAI) will be used to assess indexes of state anxiety/distress (e.g., current subjective feelings of apprehension, tension, nervousness, and worry) before and after drug administration.

A brief battery of cognitive performance assessments will be conducted on aspects of functioning known to be sensitive to the acute effects of THC and cannabis, and which are relevant to functioning in the workplace and/or in operating a motor vehicle. All subjects will be trained on the performance tasks to a stable baseline level during the screening session. Tasks include the Digit Symbol Substitution Task (DSST): Subjects must hand type patterns presented to them on a computer screen for 90 seconds and outcomes include accuracy and total number of patterns completed in the allotted time; and a computerized Paced Serial Addition Task (PSAT): Subjects are provided a string of single digit numbers on the computer and must add the total of the prior to integers presented and respond by selecting the answer using the computer mouse on the screen, primary outcome is a summed score of the number of correct trials during the task. Recent studies in our laboratory have shown that these two tasks are sensitive to cannabis dose effects.

The THC for this study will be of GMP quality and manufactured and distributed by THC Pharm GmbH (Frankfurt, Germany) in accordance with federal regulations. THC Pharm GmbH will supply pure THC in a resinous form. The THC will be suspended in pharmacy-grade ethanol (190 proof) to create a solution that is approximately 10% THC/90% ethanol (this dilution will increase the ease and precision of dosing measurements for THC, given the small doses that will be used). The ethanol solution will be purchased from Spectrum Chemical (product code: ET108), and meets standards for use in drug preparations intended in humans. Prior to pulmonary administration, the ethanol will be dissipated from the heating pad used for THC administration. The Mighty Medic is an approved medical device for administration of THC in Germany and Canada and can reliably deliver cannabinoids with similar effectiveness to the Volcano Medic device (made by the same manufacturer), which has been used to deliver THC in research studies elsewhere (including in the inventor's laboratory).

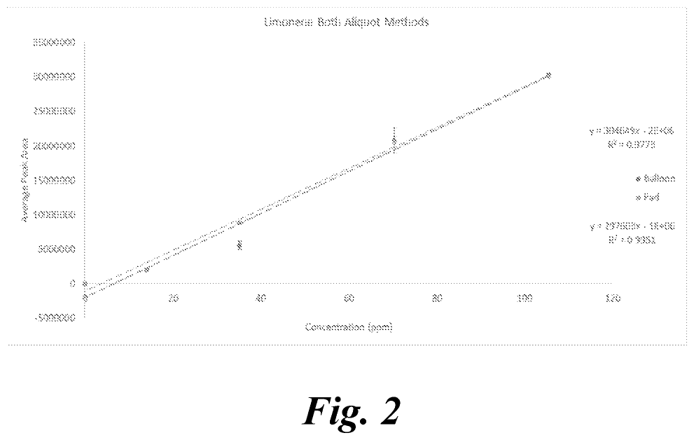

The d-limonene for this study is >99% purity, meets GMP specifications, and will be obtained from True Terpenes. Analytical testing conducted by Brian Thomas, PhD, at RTI International indicates that the Volcano Medic reliably delivers d-limonene in a linear dose-response manner, and that recovery of d-limonene from the Volcano Medic balloons via GC/MS has little variability and does not differ whether the terpene is injected directly into the balloon and equilibrated with laboratory air or vaporized at 210° C. using the Volcano Medic as will be done in the current study (see FIG. 2 for triplicate testing via both methods). Given that the Mighty Medic and Volcano Medic are demonstrated to deliver comparable levels of cannabinoids and given that the same temperature settings (210° C.) for the Mighty Medic will be used to vaporize THC and limonene, these analytical test results for the Volcano Medic are generalizable to the Mighty Medic.

The selection of doses was conducted to balance the study aim, subject safety and tolerability based on previous experience, and ensuring that doses are ecologically valid. In the inventors' laboratory, tolerant daily cannabis users have safely self-administered up to three grams of smoked cannabis containing 10% THC (300 mg THC) within one hour. More recently, acute oral, smoked and vaporized administration of 10-mg and 25-mg THC to infrequent cannabis users resulted in dose-dependent drug effects. At the 10-mg dose, subjects reported an increase in pleasant drug effects with few unpleasant side effects. At the 25-mg dose, there was no change in ratings of pleasant drug effects, but unpleasant drug effects, including self-reported ratings of anxiety, were significantly increased. Because the aim of the present study is to evaluate whether d-limonene can attenuate THC-induced anxiety, the selected THC doses represent what are likely to a good range from which to evaluate these effects. The low THC dose (15 mg) is intermediate to the 10 mg (no anxiety) and 25 mg (mild to moderate anxiety) doses we used in our recent protocol and is expected to serve as a positive control dose, although low levels of anxiety may be observed. The 30-mg dose is expected to produce anxiety in most study subjects, but not severe anxiety in the target study population.

If initial testing in this study indicates difficulty with dose tolerability, or a lack of anxiety at either dose, then we will revise the proposed doses accordingly. Adverse events beyond anxiety, sedation, or nausea (vomiting in rare instances) are unlikely given the relatively safe pharmacological profile of THC (partial agonist), which has no history of being directly associated with fatalities. In cases where a participant experiences panic and or paranoid reactions, research staff will engage the subject in relaxation exercises and will suspend research procedures until the volunteer has regained comfort. These types of effects are typically of short duration and our staff is well practiced in helping manage these types of effects.

The d-limonene doses employed in this study (1 mg, 5 mg, 15 mg) were derived based on previous experiments outlined in the background (cf. Falk et al., 1990; Falk-Filipsson et al., 1993) and the known ratios of terpenes and THC in cannabis currently being used in legal markets in the U.S. and Canada. We are not aware of any serious adverse events occurring after inhalation of d-limonene. It is commonly inhaled in ambient air due to exposure to citrus fruits, household cleaners or air fresheners, candles, or a variety of other products containing citrus essential oils.

The Mighty Medic is an approved medical device in the European Union, Canada and Israel for inhalation of THC or cannabis. The rationale behind its use is to volatilize cannabinoids and cannabis terpenoids at a temperature below that which combusts the material, and produces polyaromatic hydrocarbons (Abrams et al. 2007, Hazekamp et al. 2006, Zuurman et al. 2008).

THC is FDA approved as an oral formulation (dronabinol; Marinol©). D-limonene is Generally Recognized as Safe (GRAS) for oral consumption. As detailed above, it is thought that the THC and d-limonene administration in this study will be safe for cannabis-experienced subjects. The route of administration (vaporization) is common for cannabis self-administration and the doses of both THC and d-limonene are within the range of what would be expected in real-world cannabis use scenarios.

In sum, the presently disclosed subject matter is directed to gaining knowledge regarding the pulmonary inhalation of THC and d-limonene alone and in combination. This knowledge will advance the basic scientific understanding of both substances and may guide policy and regulations related to cannabis. The study also will extend the extant literature investigating the acute dose effects of inhaled THC, including subjective effects, cognitive performance, and their correlation with biological cannabinoid levels. These experiments may objectively demonstrate synergy of cannabis components and the modulating and even beneficial effects of cannabis terpenoids on THC. The results could benefit the research community in finding and developing selected cannabis chemovars with unusual cannabinoid and terpenoid contents and ratios for improved efficacy and optimized therapeutic index. The outcomes of this study also will be useful to inform clinical decision making and policy regarding the use of cannabis versus dronabinol, and will provide needed empirical data to either support or refute claims made in the cannabis industry that limonene mitigates the anxiogenic effects of acute, high doses of THC.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Abrams, D. I., H. P. Vizoso, S. B. Shade, C. Jay, M. E. Kelly, and N. L. Benowitz. 2007. “Vaporization as a smokeless cannabis delivery system: a pilot study.” Clin Pharmacol Ther 82 (5):572-8.
Bloor, R. N., T. S. Wang, P. Spanel, and D. Smith. 2008. “Ammonia release from heated ‘street’ cannabis leaf and its potential toxic effects on cannabis users.” Addiction 103 (10):1671-7.
Bowles, E. J. 2003. The chemistry of aromatherapeutic oils. 3rd ed. Crow's Nest, NSW, Australia: Allen & Unwin.
Buchbauer, G. 2010. “Biological activities of essential oils.” In Handbook of essential oils: science, technology, and applications., edited by K. H. C. Baser and G. Buchbauer, 235-280. Boca Raton, FL: CRC Press.
Buchbauer, G., L. Jirovetz, W. Jager, C. Plank, and H. Dietrich. 1993. “Fragrance compounds and essential oils with sedative effects upon inhalation.” J Pharm Sci 82 (6):660-4.
Carvalho-Freitas, M. I., and M. Costa. 2002a. “Anxiolytic and sedative effects of extracts and essential oil from Citrus aurantium L.” Biol Pharm Bull 25 (12):1629-33.
Carvalho-Freitas, M. I., and M. Costa. 2002b. “Anxiolytic and sedative effects of extracts and essential oil from Citrus aurantium L.” Biological and Pharmaceutical Bulletin 25 (12):1629-33.
Douthwaite, A. H., Choice of Drugs in the Treatment of Duodenal Ulcer, British Medical Journal, 4514: 43-47 (1947).
D'Souza, D. C., E. Perry, L. MacDougall, Y. Ammerman, T. Cooper, Y. T. Wu, G. Braley, R. Gueorguieva, and J. H. Krystal. 2004. “The psychotomimetic effects of intravenous delta-9-tetrahydrocannabinol in healthy individuals: implications for psychosis.” Neuropsychopharmacology 29 (8):1558-72.
Falk-Filipsson, A., A. Lof, M. Hagberg, E. W. Hjelm, and Z. Wang. 1993. “d-limonene exposure to humans by inhalation: uptake, distribution, elimination, and effects on the pulmonary function.” J Toxicol Environ Health 38 (1):77-88.
Favrat, B., A. Menetrey, M. Augsburger, L. Rothuizen, M. Appenzeller, T. Buclin, M. Pin, P. Mangin, and C. Giroud. 2005. “Two cases of “cannabis acute psychosis” following the administration of oral cannabis.” BMC Psychiatry 5 (1):17.
Felch, L. J., M. E. Di Marino, and R. R. Griffiths. 1996. “A meta-analysis of psychomotor, subject- and observer-rated effects in abuse liability studies of anxiolytics.” In Problems of drug dependence, 1995., edited by L. S. Harris, 188. Washington, D.C.: Government Printing Office.
Fukumoto, S., A. Morishita, K. Furutachi, T. Terashima, T. Nakayama, and H. Yokogoshi. 2008. “Effect of flavour components in lemon essential oil on physical or psychological stress.” Stress and Health 24:3-12.
Gaoni, Y., and R. Mechoulam. 1964. “Isolation, structure and partial synthesis of an active constituent of hashish.” J Am Chem Soc 86:1646-1647.
Haney, M., R. J. Malcolm, S. Babalonis, P. A. Nuzzo, Z. D. Cooper, G. Bedi, K. M. Gray, A. McRae-Clark, M. R. Lofwall, S. Sparenborg, and S. L. Walsh. 2016. “Oral Cannabidiol does not Alter the Subjective, Reinforcing or Cardiovascular Effects of Smoked Cannabis.” Neuropsychopharmacology 41 (8):1974-82.
Hazekamp, A., R. Ruhaak, L. Zuurman, J. van Gerven, and R. Verpoorte. 2006. “Evaluation of a vaporizing device (Volcano) for the pulmonary administration of tetrahydrocannabinol.” J Pharm Sci 95 (6):1308-17.
Ilan, A. B., A. Gevins, M. Coleman, M. A. ElSohly, and H. de Wit. 2005. “Neurophysiological and subjective profile of marijuana with varying concentrations of cannabinoids.” Behav Pharmacol 16 (5-6):487-96.
Kim, Y. W., M. J. Kim, B. Y. Chung, Y. Bang du, S. K. Lim, S. M. Choi, D. S. Lim, M. C. Cho, K. Yoon, H. S. Kim, K. B. Kim, Y. S. Kim, S. J. Kwack, and B. M. Lee. 2013. “Safety evaluation and risk assessment of d-Limonene.” J Toxicol Environ Health B Crit Rev 16 (1):17-38.
Komiya, M., T. Takeuchi, and E. Harada. 2006. “Lemon oil vapor causes an anti-stress effect via modulating the 5-HT and DA activities in mice.” Behav Brain Res 172 (2):240-9.
Komori, T., R. Fujiwara, M. Tanida, J. Nomura, and M. M. Yokoyama. 1995. “Effects of citrus fragrance on immune function and depressive states.” Neuroimmunomodulation 2 (3):174-80.
McPartland, J. M., and E. B. Russo. 2014. “Non-phytocannabinoid constituents of cannabis and herbal synergy.” In Handbook of Cannabis, edited by R. G. Pertwee, 280-295. Oxford, UK: Oxford University Press.
McPartland, J. M., and E. B. Russo. 2001. “Cannabis and cannabis extracts: Greater than the sum of their parts?” Journal of Cannabis Therapeutics 1 (3-4):103-132.
Noma, Y., and Y. Asakawa. 2010. “Biotransformation of monoterpenoids by microorganisms, insects, and mammals.” In Handbook of essential oils: science, technology, and applications., edited by K. H. C. Baser and G. Buchbauer, 585-736. Boca Raton, FL: CRC Press.
Pultrini Ade, M., L. A. Galindo, and M. Costa. 2006. “Effects of the essential oil from Citrus aurantium L. in experimental anxiety models in mice.” Life Sci 78 (15):1720-5.
Russo, E. B. 2011. “Taming THC: potential cannabis synergy and phytocannabinoid-terpenoid entourage effects.” Br J Pharmacol 163 (7):1344-64.
Russo, E. B., and G. W. Guy. 2006. “A tale of two cannabinoids: the therapeutic rationale for combining tetrahydrocannabinol and cannabidiol.” Medical Hypotheses 66 (2):234-246.
Von Burg, R. 1995. “Toxicology Update. Limonene.” Journal of Applied Toxicology 15 (6):495-499.
Zuurman, L., C. Roy, R. C. Schoemaker, A. Hazekamp, J. den Hartigh, J. C. Bender, R. Verpoorte, J. L. Pinquier, A. F. Cohen, and J. M. van Gerven. 2008. “Effect of intrapulmonary tetrahydrocannabinol administration in humans.” J Psychopharmacol 22 (7):707-16.
U.S. Pat. No. 10,588,870 for CANNABINOID FORMULATIONS FOR INHALATION, to Eck et al., issued Mar. 17, 2020.
U.S. Pat. No. 10,555,928 for CANNABIS EXTRACTS AND METHODS OF PREPARING AND USING SAME, to Blackmon et al., issued Feb. 11, 2020.
U.S. Patent Application Publication No. 20200261404 for TERPENE-ENRICHED CANNABINOID COMPOSITION AND METHOD OF TREATMENT, to Raz et al., published Aug. 20, 2020.
U.S. Patent Application Publication No. 20200101034 for RAPID AND CONTROLLED DELIVERY OF COMPOSITIONS WITH RESTORED ENTOURAGE EFFECTS, to Leone-Bay et al., published Apr. 2, 2020.

Although the foregoing subject matter has been described in some detail by way of illustration and example for purposes of clarity of understanding, it will be understood by those skilled in the art that certain changes and modifications can be practiced within the scope of the appended claims.

1. A non-naturally occurring composition comprising a combination of tetrahydrocannabinol (THC) and limonene.

2. The composition of claim 1, wherein the THC comprises a synthetic THC.

3. The composition of claim 2, wherein the synthetic THC comprises dronabinol.

4. The composition of claim 1, wherein the THC and limonene are present in the composition in a ratio of about 2:1 (THC:limonene).

5. The composition of claim 1, comprising between about 1 mg to about 50 mg THC.

6. The composition of claim 1, comprising between about 0.5 mg to about 25 mg limonene.

7. The composition of claim 1, wherein the composition of THC and limonene is selected from the group consisting of 15 mg THC:1 mg limonene; 30 mg THC:1 mg limonene; 15 mg THC:5 mg limonene; 30 mg THC:5 mg limonene; and 30 mg THC:15 mg limonene.

8. The composition of claim 1, further comprising a pharmaceutically acceptable carrier.

9. The composition of claim 8, wherein the pharmaceutically acceptable carrier is selected from the group consisting of propylene glycol, glycerin, and sesame seed oil.

10. The composition of claim 1, wherein the composition is formulated in a gelcap.

11. The composition of claim 10, wherein the composition comprises from about 1 mg to about 30 mg of THC and from about 1 mg to about 1,000 mg of limonene.

12. The composition of claim 1, wherein the composition comprises a formulation for oromucosal delivery.

13. The composition of claim 12, wherein the formulation for oromucosal delivery comprises a medium-chain triglyceride (MCT) oil, a breath strip, or an oromucosal spray.

14. A method for reducing or mitigating paranoia and/or one or more anxiogenic effects of tetrahydrocannabinol (THC), the method comprising administering to a subject in need of treatment thereof a non-naturally occurring composition of claim 1.

15. The method of claim 14, wherein the one or more anxiogenic effects comprises THC-induced anxiety.

16. (canceled)

17. The method of claim 15, wherein administering the combination of THC and limonene lowers a level of THC-induced anxiety and/or paranoia in the subject relative to a level of THC-induced anxiety and/or paranoia experienced by the subject when administered THC alone.

18. The method of claim 14, wherein the non-naturally occurring composition comprising a combination of tetrahydrocannabinol (THC) and limonene is administered orally, by vaporization, or via oromucosal delivery.

19. A method for treating one or more conditions or symptoms in a subject in need of treatment thereof, the method comprising administering a therapeutically effective amount of a non-naturally occurring composition of claim 1 to the subject to treat the one or more conditions, wherein the one or more conditions or symptoms is selected from the group consisting of arthritis, osteoarthritis, arthralgia, joint pain, joint stiffness, diabetes, lack of appetite, anorexia, vomiting, nausea, an inflammatory bowel disease including Crohn's disease and ulcerative colitis, dementia, memory loss, osteoporosis, fatigue, weakness, decreased mental energy, decreased physical energy, dizziness, deficits in balance, itchy skin, pruritus and/or chronic pruritus, wrinkles, stretch marks, skin burn, headache, migraine, weight gain, digestive problem, intestinal disorder, gastrointestinal pain, abdominal pain, pelvic pain, constipation, diarrhea, hot flashes, sweating, difficulty in concentration, weakened immune system, cardiovascular disease, palpitation and tachycardia, psoriasis, dermatophytes, Candida, leishmaniasis, Methicillin-resistant Staphylococcus aureus (MRSA), malaria, allergy, fibromyalgia, nociceptive pain, neuropathic pain, pain, arm or leg pain, acne, insomnia and/or sleep disorders, muscle pain, myalgia, spasticity, muscle tension, cramps, spasms, anxiety, irritability, nervousness, restless, stress, depression, mood problem, affect disorders, anger, autism and/or autism spectrum disorder, neurodegenerative diseases, Alzheimer disease, Parkinson disease, Huntington's disease, Dystonia, Amyotrophic Lateral Sclerosis (ALS), multiple sclerosis, Tourette syndrome, Myasthenia Gravis, epilepsy, symptomatic treatment of cancer, primary treatment of cancer, inflammatory airway diseases including asthma and/or chronic obstructive pulmonary disease (COPD), addiction, stroke, traumatic brain injury, motor spasm and/or tic, vocal tic, obsessive compulsive disorder (OCD), attention deficit hyperactivity disorder (ADHD), attention deficit disorder (ADD), cerebral palsy (CP), inflammation, oxidative stress, paresthesia, glaucoma, heartburn, pyrosis, gastric ulcers and H. pylori infection, and combinations thereof.

20. (canceled)

21. The method of claim 19, wherein the one or more conditions or symptoms is selected from the group consisting of anorexia associated with weight loss in subjects afflicted with AIDS, nausea and vomiting associated with cancer chemotherapy in subjects who have failed to respond to conventional antiemetic treatments, and sleep apnea.

22. The method of claim 15, wherein the non-naturally occurring composition comprising a combination of tetrahydrocannabinol (THC) and limonene is administered orally, by vaporization, or via oromucosal delivery.
